(19) United States
(12) Patent Application Publication
(10) Pub. No.: US 2012/0184774 A1
(43) Pub. Date: Jul. 19, 2012

(54) PROCESS FOR THE PREPARATION OF PHARMACEUTICALLY ACCEPTABLE SALTS OF RACEMIC MILNACIPRAN AND ITS OPTICAL ENANTIOMERS THEREOF

(75) Inventors: Vikram Sarjerao Jagtap, Mumbai (IN); Ravindra Baburao Chache, Thane (W) (IN); Kamiesh Jayantital Ranbhan, Thane (IN); Yuvaraj Kashinath Zunjarrao, Thane (E) (IN); Pushpalata Sarjekar, Thane (IN); Arun Kanti Mandal, Mumbai (IN); Ganesh Gurpur Pai, Thane (IN)

(73) Assignee: Arch Pharmalabs Ltd.

(21) Appl. No.: 13/330,967

(22) Filed: Dec. 20, 2011

(30) Foreign Application Priority Data
Jan. 14, 2011 (IN) 124/MUM/2011

(51) Int. Cl. C07C 231/12

(52) U.S. Cl. 564/164

(57) ABSTRACT

The present invention relates to an improved process for the preparation of pharmaceutically acceptable salts of milnacipran by mutual acid radical exchange.

This application claims priority of India Application Number 124/MUM/2011 filed on Jan. 14, 2011 and is included herein in its entirety by reference.

### COPYRIGHT NOTICE

A portion of the disclosure of this patent contains material that is subject to copyright protection. The copyright owner has no objection to the reproduction by anyone of the patent document or the patent disclosure as it appears in the Patent and Trademark Office patent files or records, but otherwise reserves all copyright rights whatsoever.

### BACKGROUND OF THE INVENTION

1. Field of the Invention

The present invention relates to an improved process for the preparation of pharmaceutically acceptable salts of milnacipran by mutual acid radical exchange. Herein the term “milnacipran” includes racemic milnacipran and its optical enantiomers unless specified. The process comprises the treatment of the racemic milnacipran or mixture of its enantiomers or optical enantiomer with an organic acid which generates the corresponding less soluble acid addition salt that is either purified or is enriched. Recrystallization or pulping using various organic solvents allows either purification or enrichment of the specific enantiomer. The said organic acid salts of the cis-milnacipran or its optical enantiomers prepared according to the present invention could be further converted into the pharmaceutically acceptable salt directly in a single step by exchange of the organic acid salt with mineral acid radical avoiding generally practiced additional process step of the generation of free base which is then converted into the desired pharmaceutically acceptable salt.

2. Description of Related Art

Racemic milnacipran chemically named as 1-phenyl-2-(aminomethyl)cyclopropane-N,N-diethyl carboxamide) was first approved for the treatment of major depressive episodes in France in December 1996. It is currently marketed (as Ixel) for this indication in over 45 countries worldwide including several European countries such as Austria, Bulgaria, Finland, France, Portugal, and Russia. It is also available in Japan (as Toledomin) and Mexico (as Dalcipran). Cypress Bioscience bought the exclusive rights for approval and marketing of the drug for any purpose in the United States and Canada in 2003 from the inventor Pierre Fabre. It is reported that said drug is also used to treat fatigue, pain, fibromyalgia, irritable bowel syndrome, and the like. At present, it is mostly sold in the form of racemic cis milnacipran hydrochloride. Milnacipran belongs to dual inhibitors of serotonin and norepinephrine reuptake (SNRI), which is the fourth generation antidepressant and can inhibit both serotonin and norepinephrine reuptakes, with similar action strength. It is mainly useful to treat depression. Currently 22 countries have approved racemic cis milnacipran for treating depression. Cis milnacipran (Z(±)-2-(amino methyl)-N,N-diethyl-1-phenyl cyclopropane carboxamide), a molecule synthesized at the PIERRE FABRE MEDICAMENT Research Centre (Castres, France), also known as TN-912, dalcipran, minalcipran, midalcipran or midalipran is known to be a dual inhibitor of serotonin (5-HT) and norepinephrine (NE) reuptake. Dual inhibitors of serotonin (5-HT) and norepinephrine (NE) reuptake correspond to a well-known class of antidepressant agents which selectively inhibit reuptake of both serotonin and norepinephrine. By way of example, venlafaxine and duloxetine are also dual inhibitors of serotonin and norepinephrine. Studies have shown that the ratio of norepinephrine reuptake inhibition to serotonin reuptake inhibition by cis milnacipran is approximately 2:1 (Moret et al., 1985 Neuropharmacology 24(12): 1211-1219; Palmier et al., 1989, Eur J Clin Pharmacol 37: 235-238).

In January 2009 the U.S. Food and Drug Administration (FDA) approved racemic cis milnacipran (under the brand name Savella) for the treatment of fibromyalgia, making it the third medication approved for this purpose in the United States. Fibromyalgia, which is estimated to affect from 2-4% of the population in the US, is a complex syndrome associated with chronic widespread musculoskeletal pain and a reduced pain threshold, with hyperalgesia and allodynia (pain-related behavior in response to normally innocuous stimuli). Some associated clinical features include fatigue, depression and other mood disorders, anxiety, sleep disturbances, headache (including migraines), changes in bowel habits (including irritable bowel syndrome), diffuse abdominal pain, and urinary frequency.

The study of racemic cis milnacipran has revealed that the medication containing cis milnacipran does not interact with other medications as it has lower ability of plasma protein-binding, moreover cis milnacipran's half-life is relatively shorter, it has an advantage of no residual effect after treatment, and therefore it has fine tolerance and security, thus it has acquired a lot of importance in the present scenario.

In 1992, a resolution had been approved by American Food and Drug Administration (FDA) and The European Committee for Proprietary Medicinal Products, which encouraged that drugs with chiral center should be in optically pure form for marketing authorization; in 1996, a project had been proposed by FDA that drugs with chiral center must be in optically pure form when it is applying for marketing authorization. There are two chiral centers in the molecular structure of milnacipran; there should be two groups of enantiomers and therefore four compounds in theory. Due to the molecular configuration, the cis-isomer is the main synthetic product, that exists in two forms of optical enantiomers: the dextrogyral enantiomer of cis-milnacipran hydrochloride Z-(1S,2R) of formula IV chemically named as Z-(1S,2R)-2-(amino methyl)-N,N-diethyl-1-phenyl cyclopropane carboxamide and the levogyral enantiomer of cis-milnacipran hydrochloride Z-(1R,2S) of formula V chemically named as Z-(1R,2S)-2-(amino methyl)-N,N-diethyl-1-phenyl cyclopropane carboxamide. In its hydrochloride form, cis milnacipran (also called F2207) is currently marketed as Lexel in the form of a racemic mixture as a serotoninergic and norepinephrinergic antidepressant agent. F2695 and F2696 represent the dextrogyral and levogyral enantiomers respectively of cis milnacipran hydrochloride (F2207).

Cis milnacipran and its method of preparation are described in U.S. Pat. No. 4,478,836. The said patent also describes the use of cis milnacipran for the treatment of disorders of the central nervous system, in particular depression.

The references US2004/0162334, US20060014837, CN1699332A, and U.S. Pat. No. 7,005,452 disclose the use of a mixture of enantiomers enriched in the dextrogyral enantiomer of cis milnacipran as well as their pharmaceutically-acceptable salts, for the preparation of a drug intended to prevent or to treat disorders that can be managed by double inhibition of serotonin (5-HT) and norepinephrine (NE) reuptake, while limiting the risks of cardiovascular disturbances and/or organ and/or tissue toxicity. The said patent applications also disclose that the dextrogyral enantiomer of cis milnacipran hydrochloride had activity which was significantly higher than racemic cis milnacipran, with less risk of cardiovascular disturbances and tissue and organ organic toxicity. However, the said patent or applications do not disclose any information related to pharmacology of levogyral enantiomer of cis milnacipran.

European journal of metabolism and pharmacokinetics, 1998, April-June, 23(2), 166-171 discloses that both dextrogyral referred as F2695 and levogyral referred as F2696 are pharmacologically active, although F2695 carries most of the pharmacology activity.

U.S. Pat. No. 7,074,833 clearly discloses that dextrogyral referred as F2695 is two times more superior than the racemic milnacipran and twenty-five times more than levogyral, referred as F2696. Moreover, it also discloses that F2696 is more toxic than F2695. It also discloses the use of dextrogyral F2695 for the preparation of medicine for disorders.

FR2581060B1 discloses an industrial process for the preparation of milnacipran hydrochloride as per scheme-I given herein below:

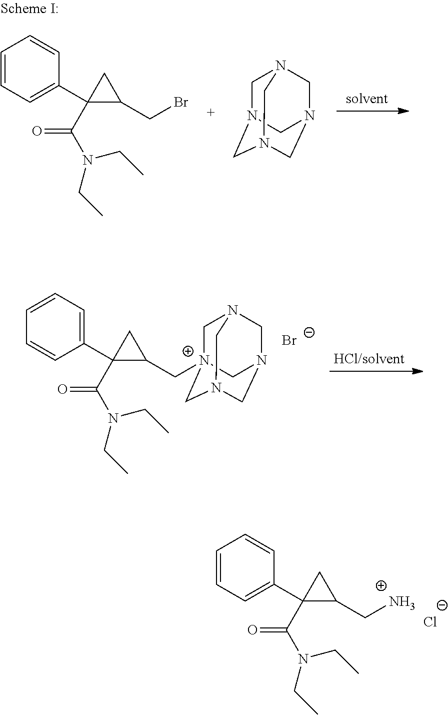

EP200638B1 discloses a process comprising reaction of cis-1-phenyl-1-diethylcarbamoyl-2-phthalimidomethyl-cyclopropane with a primary alkylamine or hydroxyalkylamine, optionally in a solvent, to get (Z)-1-phenyl-1-diethylaminocarbonyl-2-(aminomethyl)cyclopropane base followed by its reaction with HCl to give (Z)-1-phenyl-1-diethylaminocarbonyl-2-(aminomethyl)cyclopropane hydrochloride. Schematic representation of the above process is depicted in the scheme II.

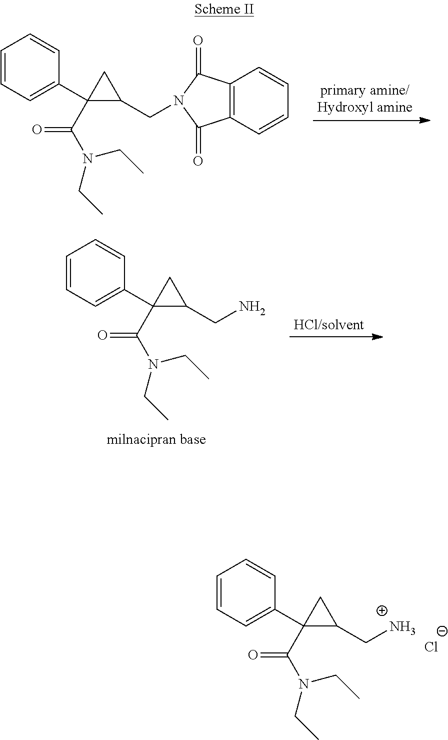

US20100016636A1 (hereinafter referred as '636) discloses racemic milnacipran and tartaric acid derivatives or their compositions used as resolving agents are dissolved in ketone or alcohol solvent. Crystallization is performed under room temperature and diastereoisomer salt is precipitated and filtered. The resolved product is separated and suspended in organic solvent and water. The tartaric acid salt is treated with an alkali to obtain optically pure (Z)-1-phenyl-1-diethylaminocarbonyl-2-(aminomethyl)-cyclopropane free base. The free base is reacted with an acid to obtain its corresponding salt. '636 discloses that to prepare pharmaceutically active HCl salt of (Z)-1-phenyl-1-diethylaminocarbonyl-2-(aminomethyl)-cyclopropane, the optically pure organic acid salt has to be hydrolysed to generate a free base which in the second process step is converted into the pharmaceutically active salt. There are three process steps to convert the racemic mixture of a compound into the acid addition salt of optically pure compound of the said racemic mixture viz:
i) Preparation of organic acid addition salt of the racemic mixture of with an optically pure tartaric acid derivative;
ii) after the isolation of the optically pure tartaric acid derivative salt the said organic salt is hydrolyzed to obtain free base;
iii) free base is then treated with the mineral acid to obtain inorganic acid addition salt.
   '636 neither teaches nor motivates a person skilled in the art to convert the organic acid addition salt which is a tartaric acid derivative salt in this case directly into the mineral acid salt without hydrolysis process step to isolate the free base.

JP04354436B2 discloses a process for preparing milnacipran hydrochloride comprising reaction of (Z)-1-phenyl-1-diethylaminocarbonyl-2-(hydroxymethyl)-cyclopropane with sulphonyl halide to obtain corresponding sulphonate. This sulphonate is then reacted with hexamethylene tetramine, to obtain (Z)-1-phenyl-1-diethylamino carbonyl-2-hexamethylene tetra ammonium methyl cyclopropane salt. The obtained salt is then processed with hydrochloric acid, to obtain milnacipran hydrochloride. Schematic representation of the above process is depicted in the scheme III.

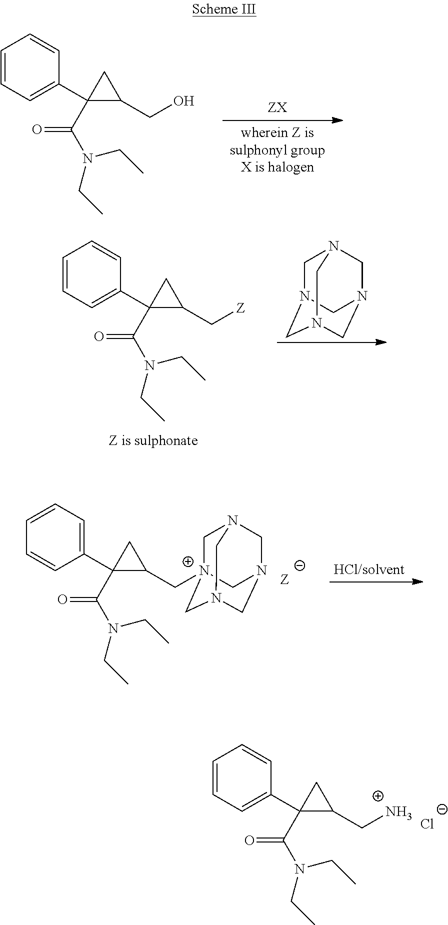

The said patent uses sulphonyl halide followed by its hexamethylenetetramine salt preparation but it neither teaches nor motivates the use of the acid for the corresponding acid salts, which are easy to form due to the presence of free amino group in the milnacipran molecule. Disadvantage of the said prior art is the use of sulphonyl halides which are fumigating.

Therefore, there is not any prior art which discloses, teaches or motivates a person skilled in the art about the direct conversion of acid addition salt of milnacipran into milnacipran hydrochloride without going through the process step of hydrolysis of the organic acid addition salt which is prepared either to purify impure racemic mixture milnacipran base by converting into an organic acid addition salt or for resolution by treating a racemic mixture with an optically active organic salt.

### BRIEF SUMMARY OF THE INVENTION

The present invention relates to an improved process for the preparation of pharmaceutically acceptable salts of milnacipran by mutual acid radical exchange. Herein the term “milnacipran” includes racemic milnacipran and its optical enantiomers. The process comprises the treatment of the racemic milnacipran or mixture of its enantiomers with an organic acid which generates a less soluble acid addition salt that is either purified or is enriched. Recrystallization or pulping by standard crystallization process allows either purification or enrichment of the specific enantiomer. The organic acid salts of the racemic cis-milnacipran or its optical enantiomers prepared according to the present invention could be further converted into the mineral acid salt by exchange of the organic acid salt with mineral acid in a single step directly without hydrolysis process step to isolate free base.

### BRIEF DESCRIPTION OF THE DRAWINGS

FIG. 1 is a minacpran salt wherein A is anionic part of an acid.

FIG. 2 is a Racemic form of (cis)milnacipran.

FIG. 3 is a (1S,2R)-(cis)-milnacipran.

FIG. 4 is a (1R,2S)-(cis)-milnacipran.

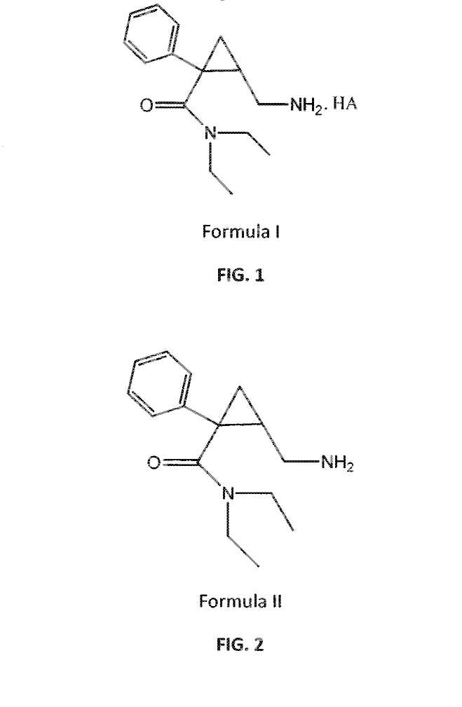

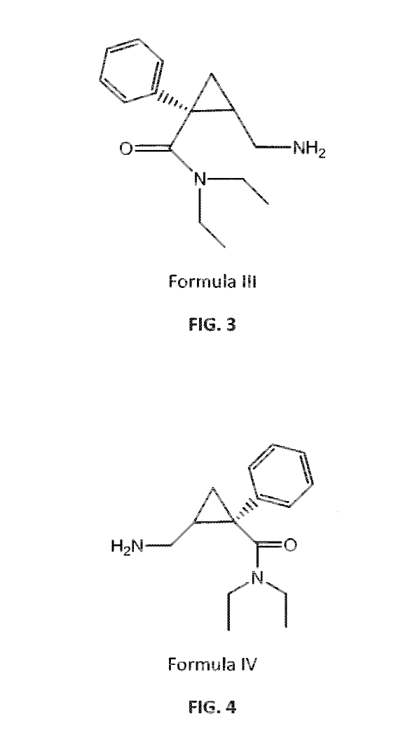

### DETAILED DESCRIPTION OF THE INVENTION

While this invention is susceptible to embodiment in many different forms, there is shown in the drawings and will herein be described in detail specific embodiments, with the understanding that the present disclosure of such embodiments is to be considered as an example of the principles and not intended to limit the invention to the specific embodiments shown and described. In the description below, like reference numerals are used to describe the same, similar or corresponding parts in the several views of the drawings. This detailed description defines the meaning of the terms used herein and specifically describes embodiments in order for those skilled in the art to practice the invention.

The terms “a” or “an”, as used herein, are defined as one or as more than one. The term “plurality”, as used herein, is defined as two or as more than two. The term “another”, as used herein, is defined as at least a second or more. The terms “including” and/or “having”, as used herein, are defined as comprising (i.e., open language). The term “coupled”, as used herein, is defined as connected, although not necessarily directly, and not necessarily mechanically.

The term “about” means±10 percent.

The term “substantially” means±10 percent.

Reference throughout this document to “one embodiment”, “certain embodiments”, and “an embodiment” or similar terms means that a particular feature, structure, or characteristic described in connection with the embodiment is included in at least one embodiment of the present invention. Thus, the appearances of such phrases or in various places throughout this specification are not necessarily all referring to the same embodiment. Furthermore, the particular features, structures, or characteristics may be combined in any suitable manner in one or more embodiments without limitation.

The term “or” as used herein is to be interpreted as an inclusive or meaning any one or any combination. Therefore, “A, B or C” means any of the following: “A; B; C; A and B; A and C; B and C; A, B and C”. An exception to this definition will occur only when a combination of elements, functions, steps or acts are in some way inherently mutually exclusive.

The drawings featured in the figures are for the purpose of illustrating certain convenient embodiments of the present invention, and are not to be considered as limitation thereto. Term “means” preceding a present participle of an operation indicates a desired function for which there is one or more embodiments, i.e., one or more methods, devices, or apparatuses for achieving the desired function and that one skilled in the art could select from these or their equivalent in view of the disclosure herein and use of the term “means” is not intended to be limiting.

Present invention relates to an industrially acceptable improved process for the preparation of milnacipran pharmaceutically acceptable salts of formula I wherein A is anionic part of an acid and also for its optical enantiomers namely dextrogyral Z(1S,2R) of formula III ((1S,2R)-(cis)-milnacipran) and levogyral Z(1R,2S) of formula IV ((1R,2S)-(cis)-milnacipran) from corresponding racemic mixture (50:50 of dextrogyral: levogyral) or mixture of dextrogyral and levogyral enantiomers or dextrogyral and levogyral enantiomers. Its Racemic form Formula II (Racemic form of (cis) milnacipran) is also shown.

The present invention not only reduces a process step and minimizes unit operation but also minimizes yield loss by avoiding isolation of milnacipran base and also improves the purity.

The present invention discloses a process for the preparation of acid addition salt of milnacipran with organic acid and their conversion directly into the pharmaceutically acceptable salts which are formed by mutual exchange of acid radical, in one embodiment, by mineral acid selected from hydrochloric, hydrobromic, hydrogeniodic, sulphuric acid, phosphoric acid and the like. Herein above and herein below, the term “milnacipran” means racemic as well as optical enantiomers. The present invention also relates to a process comprising organic acid salt preparation as a mode of purification of racemic milnacipran or as a mode of enrichment of optically active enantiomer of milnacipran or as a tool for achieving desired impurity profile for optically pure enantiomer. The said process results into milnacipran having an excellent chemical and enantiomeric purity.

In the case where organic acid is non chiral, it reacts with racemic milnacipran and acts as a mode of purification as it forms salt with free amino groups of the milnacipran moiety, separates out as solid material which can be easily filtered off and further optionally purified by standard crystallization method. Later this purified salt can be directly converted into its pharmaceutically acceptable salts, in one embodiment, those of mineral acid by mutual exchange of acid radically avoiding the process of hydrolysis to obtain free base as reported in the prior art.

In the case where racemic milnacipran is pure but does not qualify the criteria of impurity profile, the said invention works well by forming the organic acid addition salt of milnacipran leaving behind the impurities in mother liquor.

In the case of preparing optical enantiomers of milnacipran and their pharmaceutically acceptable salts, the present invention works in three ways depending upon the choice of acid for the purpose.

If the organic acid under the invention is optically pure, it serves three purposes as salt formation along with resolution providing salts of optical enantiomers, purification by the salt formation as it forms salt with free amino group of the resolved optical enantiomers of milnacipran and enrichment of the specific optical enantiomer by choosing (+) or (−) chiral acid. The said salt can be directly converted into its pharmaceutically acceptable salts, in one embodiment those of mineral acid by mutual radical exchange of acid in a single step.

In the case where optically active milnacipran is pure but does not qualify the criteria of impurity profile, the said invention works well by forming the organic acid addition salt of milnacipran leaving behind the impurities in mother liquor.

Disclosed herein is also a process for the preparation of optically pure enantiomer of milnacipran wherein the organic acid addition salt of racemic optically enriched or optically pure enantiomer is directly converted into the corresponding pharmaceutically active mineral acid salt by mutual exchange of organic acid radical with the desired mineral acid radical in a single step.

Reference will now be made in detail to embodiments of the invention. This invention may, however, be embodied in many different forms and should not be construed as limited to the embodiments set forth herein. In addition, and as will be appreciated by one of the skill in the art, the invention may be embodied as a method, system or process.

Efficient, industrially viable and economical process for the preparation of pharmaceutically acceptable salts of milnacipran and its optical enantiomers by the mutual acid radical exchange method is illustrated in the following reaction schemes.

Compound of formula I and its hydrochloride is known in the art. Present invention relates to an efficient process for the preparation of pharmaceutically acceptable salts over the processes known in the art to overcome the shortcomings therein in the processes disclosed in the prior art.

Racemic milnacipran required for the purpose of the present invention is prepared by the process as disclosed in EP0200638B1. The schematic representation for the same is as given below:

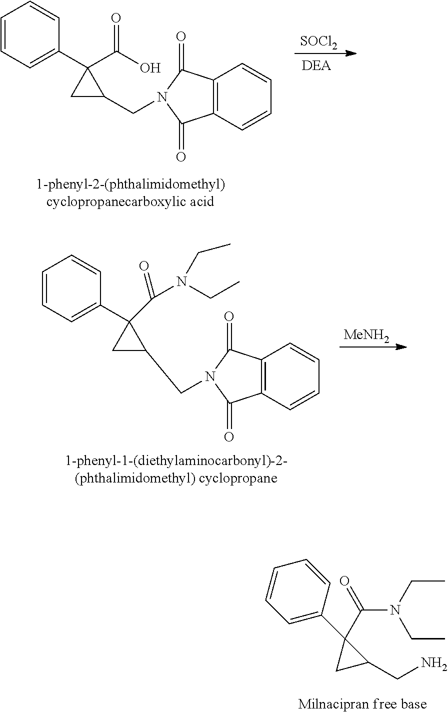

In first embodiment of the invention, racemic milnacipran is reacted with an organic acid to form the corresponding organic acid addition salt in a solvent to purify the milnacipran. The said organic acid addition salt of milnacipran so obtained is then converted into pharmaceutically acceptable salts by mutual acid radical exchange by the addition of corresponding acid. The invention is represented by scheme IV given herein below. Acid addition salt formed in this embodiment contains milnacipran with an improved chemical purity than the original milnacipran base used for the purpose. Therefore, salt formation works as one mode of purification of milnacipran. Secondly, purified salt is directly converted into pharmaceutically acceptable salt in a single step avoiding hydrolysis and isolation of purified milnacipran base by the addition of corresponding acid. Schematic representation for the said embodiment is as given in scheme IV.

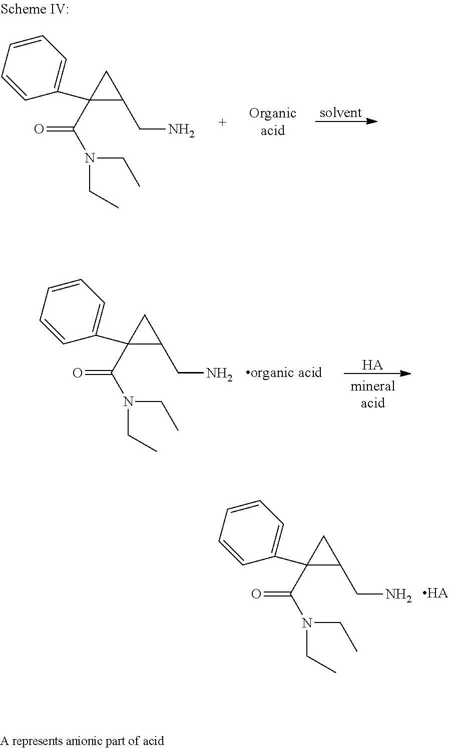

HA is mineral acid.

Racemic milnacipran defined in the first embodiment includes impure, pure milnacipran having a particular impurity profile not meeting desired specifications of the pharmaceutically acceptable milnacipran.

Organic acid used for the purpose of the first embodiment is selected from the group of organic acids which are capable of forming acid addition salts with milnacipran. In one embodiment, organic acid for the present invention is selected from citric acid, succinic acid, malic acid, mandelic acid, oxalic acid, tartaric acid, or derivatives thereof, and the like.

Solvent used for the purpose of first embodiment is chosen from the group of esters, aromatic hydrocarbon and ketones.

Acids used for the preparation of pharmaceutically acceptable salts are selected from the group of hydrochloric acid, hydrobromic acid, hydroiodic acid, sulphuric acid, phosphoric acid, and the like.

In one embodiment, racemic milnacipran having the less chemical purity is reacted with oxalic acid using ethyl acetate as a solvent to prepare racemic milnacipran oxalate containing milnacipran with enhanced chemical purity. This optionally purified salt is then reacted with hydrochloric acid using isopropyl alcohol as a solvent to prepare milnacipran hydrochloride in a single step avoiding isolation of the free base. Schematic representation for the said embodiment is as given in scheme V.

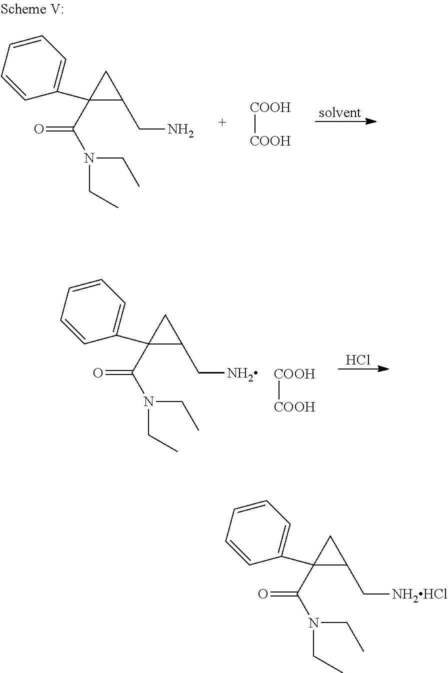

In another embodiment wherein racemic milnacipran having higher chemical purity but not meeting the impurity profile as per the requirement in the market or industries is reacted with organic acid to prepare the corresponding milnacipran organic acid addition salt. The said salt is then optionally purified and reacted with hydrochloric acid to prepare the milnacipran hydrochloride with improved impurity profile in a single step without isolating the free base.

Inventors of the present invention found that racemic milnacipran obtained by the route of synthesis that is followed remains associated with N,N′-dimethylphthalimide as an impurity up to the extent of about 5%, which is formed during deprotection of phthalimide group using monomethyl amine.

The present invention has been proven to be a successful tool for the removal of the said impurity by employing the efficient process disclosed herein in the present invention.

In another embodiment wherein racemic milnacipran acid addition salt having higher chemical purity is converted in a single step into milnacipran hydrochloride with the same chemical purity.

In the second embodiment of the invention, racemic milnacipran is reacted with chiral organic acid in a solvent to prepare diastereomeric salts of milnacipran with enhanced optical purity. The optionally purified salt is then directly converted into pharmaceutically acceptable salts in a single step by the addition of corresponding mineral acid eliminating the process steps of hydrolysis to isolate milnacipran base. The preparation of chiral acid addition salt of milnacipran is represented in scheme VI.

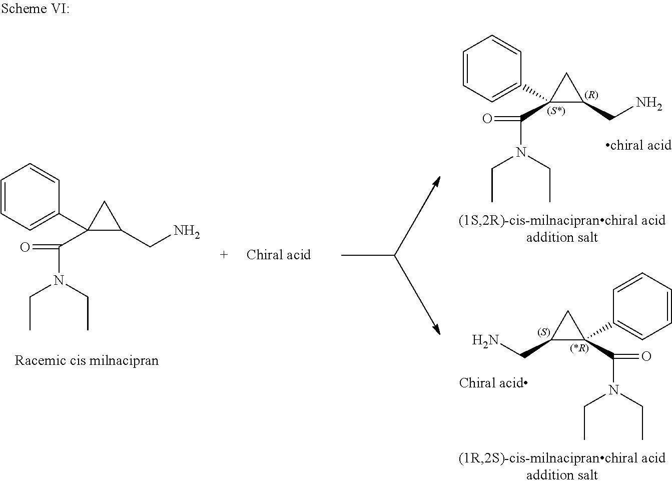

Chiral organic acid for the second embodiment of the present invention is selected from the group of tartaric acid, di-p-toluoyl tartaric acid, di-p-benzoyl tartaric acid, di-p-methoxybenzoyl tartaric acid, camphor sulphonic acid, mandelic acid and derivatives thereof and the like.

Solvent is selected from water, aromatic hydrocarbon, aliphatic alcohol, aliphatic ketone, ether, ester, acyclic and cyclic aliphatic hydrocarbon or mixture thereof. In one embodiment solvent is selected from toluene, isopropanol alcohol, acetone, methyl tert butyl ether, cyclohexane, hexane and the like. In one embodiment the solvent is water.

In one embodiment crude racemic milncipran is dissolved in water, till a clear solution is obtained followed by the addition of aqueous solution of D-(−)-mandalic acid under stirring, heat optionally, continue stirring till (1S,2R)-cis-milnacipran D-(−)-mandelate is precipitated completely. The optically pure (1S,2R)-cis-milnacipran D-(−)-mandelate so obtained is filtered off and washed with water. This isolated (1S,2R)-cis-milnacipran D-(−)-mandelete contained about 99% optically pure (1S,2R)-cis-milnacipran with yield of about 89%. Only single crystallization from ethyl acetate enhanced optical purity to about 99% with yield of about 77%. The said salt is then directly converted in a single step into (1S,2R)-cis-milnacipran hydrochloride with required purity without hydrolysis to prepare and isolate (1S,2R)-cis-milnacipran base. Schematic representation for the said embodiment is as given in scheme VII.

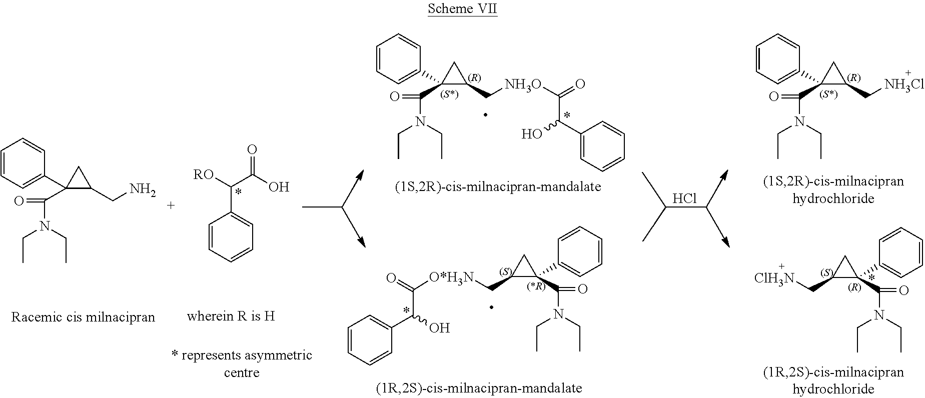

In another embodiment crude racemic milncipran is dissolved in water, till a clear solution is obtained followed by the addition of L-(+)-mandalic acid under stirring, heat optionally, continue stirring till (1R,2S)-cis-milnacipran L-(+)-mandelate is precipitated completely. The optically pure (1R,2S)-cis-milnacipran L-(+)-mandelate so obtained is filtered off and washed with water. This isolated (1R,2S)-cis-milnacipran L-(+) mandelete contained about 99% optically pure (1R,2S)-cis-milnacipran with a yield of about 74%. Only single crystallization from ethyl acetate enhanced optical purity to about 99% with yield of about 72%. The said salt is then directly converted in a single step into (1R,2S)-cis-milnacipran hydrochloride with required purity without hydrolysis to prepare and isolate (1R,2S)-cis-milnacipran base. Schematic representation for the said embodiment is as given in scheme VII.

In another embodiment of the invention, optically pure milnacipran but not meeting impurity profile criteria is reacted with organic acid to obtain organic acid addition salt of optically pure milnacipran, which is filtered off. The said organic acid addition salt is then optionally purified and is converted directly in a single step into milnacipran hydrochloride by addition of hydrochloric acid meeting the required criteria of impurity profile.

In another embodiment wherein optically active milnacipran acid addition salt having higher optical purity is converted in a single step into milnacipran hydrochloride with the same optical purity.

### EXAMPLES

The following non limiting examples are provided to illustrate further the present invention. It will be apparent to those skilled in the art that many modifications, variations and alterations to the present disclosure, both to materials, methods and reaction conditions, may be practiced. All such modifications, variations and alterations are intended to be within the spirit and scope of the present inventions. It should be understood that the present invention is not construed as being limited thereto.

### Example 1

### Process for Preparation of Racemic Milnacipran Hydrochloride from Racemic Milnacipran Oxalate Salt without Isolating Milnacipran Base

A. Racemic milnacipran (20.0 g 0.081 moles) having chemical purity of 64% was taken in 150 ml ethyl acetate and mixture was stirred to get clear solution; oxalic acid (8 g, 0.089 moles) was added to the above contents in one lot. Initial clear solution turned into precipitate after stirring for ½ hr. Contents were heated to 60-65° C. for 30 minutes and it was gradually cooled to room temperature and maintained under stirring for 4-5 hrs followed by chilling to 10-15° C. for one hour. Precipitated solid i.e. racemic milnacipran oxalate salt was filtered off. Yield was about 60% of theory and chemical purity was more than 95%.

B. Racemic milnacipran oxalate salt (10.0 g 0.029 moles) obtained from example 1 was taken in 20 ml of isopropyl alcohol and 20 ml methyl tert butyl ether followed by passing of dry HCl gas till pH less than 1. The contents were stirred for an hour to get clear solution. The mixture was slowly brought to room temperature and then at 0-5° C. for the next 6 hours. The solid was filtered off and washed with precooled mixture of IPA (15 ml) and methyl tert butyl ether (150 ml), to 5° C.

Dry weight of racemic milnacipran hydrochloride was 5.4 g. Yield is 73% of the theory.

### Example 2

### Process for Preparation of Pure Racemic Milnacipran Hydrochloride from Impure Racemic Milnacipran Oxalate Salt without Isolating Milnacipran Free Base

A. Racemic milnacipran (20.0 g 0.081 moles chemical purity of 97% but having 2.7% impurity of N,N′-dimethylphthalimide was taken in 150 ml ethyl acetate and mixture was stirred to get clear solution; oxalic acid (8 g, 0.089 moles) was added to the above contents in one lot. Initial clear solution turned into precipitate after stirring for ½ hr. Contents were heated to 60-65° C. for 30 minutes and it was gradually cooled to room temperature and maintained under stirring for 4-5 hours followed by chilling to 10-15° C. for one hour. Precipitated solid i.e. racemic milnacipran oxalate salt was filtered off. Yield was about 61% of theory and chemical purity was found to be 99.6% with the said impurity as 0.09%.

B. Racemic milnacipran oxalate salt (10.0 g 0.029 moles with 0.09% N,N′-dimethylphthalimide) obtained from example 2 was taken in 20 ml of isopropyl alcohol and 20 ml methyl tert butyl ether followed by passing of dry HCl gas till pH less than 1. The contents were stirred for an hour to get clear solution. The mixture was slowly brought to room temperature and then at 0-5° C. for next 6 hours. The solid was filtered off and washed with precooled mixture of IPA (15 ml) and methyl tert butyl ether (150 ml), to 5° C.

Dry weight of racemic milnacipran hydrochloride was 5.4 g. Yield is 71% of the theory with N,N′-dimethyl phthalimide impurity less than 0.05%.

### Example 3

### Process for Preparation of Chiral Milnacipran D(−)Hydrochloride from Chiral Milnacipran D(−)Mandelate Salt without Isolating Optical Milnacipran Free Base

A. Racemic cis-milnacipran (20.0 g 0.081 moles) is taken in 70 ml water, the mixture is stirred to get clear solution followed by the addition of D(−)mandelic acid (14.0 g, 0.092 moles) solution made in 70 ml water. The mixture is stirred, solid formation is observed, stirring is continued for 1.0 hour. Contents are heated to 60-65° C. to get clear solution and further maintained for 30 minutes to 60 minutes. The mixture is gradually brought to room temperature and maintained under stirring for 8-10 hours. The crystallized solid i.e. (1S,2R)-cis-milnacipran-(D)-mandelate salt is filtered off.

B. Chiral milnacipran D(−) mandelate salt (25.0 g, 0.063 moles) was suspended in the mixture of 20 ml of isopropyl alcohol and 65 ml of methyl tertbutyl ether. The mass was slowly heated to 40° C. At this temperature 11-13 ml of 18% IPA.HCl was added drop wise to get clear solution. The contents were stirred for ½ hr and then cooled to 0-5° C. and maintained for 4 hr. Obtained solid was filtered and washed with precooled mixture of IPA (15 ml) and methyl tert butyl ether (150 ml). The solution is vacuum treated to dryness. Dry weight 13 g. Yield is about 74% of theory and optical purity of required (+) Milnacipran contained in which 95-96% e.e.

A. Racemic cis-milnacipran (20.0 g 0.081 moles) is taken in 70 ml water, the mixture is stirred to get clear solution followed by the addition of L (+) mandelic acid (14.0 g, 0.092 moles). The mixture is stirred, solid formation is observed, stirring is continued for 1.0 hour. Contents are heated to 60-65° C. to get clear solution and further maintained for 30 minutes to 60 minutes. The mixture is gradually brought to room temperature and maintained under stirring for 8-10 hours. The crystallized solid i.e. (1R,2S)-cis-milnacipran L(+) mandelate salt is filtered off.

B. Chiral milnacipran L(+) mandelate salt (25.0 g, 0.063 moles) was suspended in the mixture of 20 ml of isopropyl alcohol and 80 ml of methyl tertbutyl ether. The mass was slowly heated to 40° C. At this temperature 11-13 ml of 18% IPA.HCl was added drop wise to get clear solution. The contents were stirred for ½ hr and then cooled to 0-5° C. and maintained for 4 hours. Obtained solid was filtered and washed with precooled mixture of IPA (15 ml) and methyl tert butyl ether (150 ml). The solution is vacuum treated to dryness. Dry weight 14 g. Yield is about 79% of theory and optical purity of 1R,2S milnacipran hydrochloride contained in which 95-96% e.e.

### OBJECT OF THE INVENTION

The first embodiment of the invention is to provide a novel process for the preparation of pharmaceutically acceptable salts of racemic milnacipran and its optical enantiomers.

The second embodiment of the invention is to provide a novel process for the preparation of pharmaceutically acceptable salts of milnacipran using milnacipran acid addition salts as an intermediate.

The third embodiment of the invention is to provide a process comprising direct conversion of acid addition salt of milnacipran into its pharmaceutically acceptable salt by mutual acid exchange without the hydrolysis of acid addition salt to isolate free base.

The forth embodiment of the present invention is the purification of racemic milnacipran having less chemical purity by its acid addition salts.

The fifth embodiment of the invention is to provide an improved process for the preparation of pharmaceutically acceptable salts of optical enantiomers of milnacipran using water as solvent.

The sixth embodiment of the invention is to skip and avoid the hydrolysis of intermediate acid addition salt to obtain free base which is then by an additional process step is converted into the desired pharmaceutically active salt.

The seventh embodiment of the invention is to avoid the loss of yield by the additional step of hydrolysis of the intermediate acid addition salt to obtain free base.

The eighth embodiment of the invention is to qualify the criteria of impurity profile by the salt preparation of organic acid addition salt which in turn in a single step is converted into pharmaceutically acceptable salt of milnacipran meeting with criteria of impurity profile.

### ADVANTAGES OF THE PRESENT INVENTION

1. The process eliminates the hydrolysis step of the intermediate acid addition salt to isolate the milnacipran base.
2. One unit operation of hydrolysis to obtain free base is reduced.
3. Acid addition salt formation acts as a mode of purification for making racemic milnacipran and its optical enantiomers.
4. Use of simple commercially available acids thereby reducing overall costs of the product.
5. Higher yield and higher purity.
6. Use of water as solvent.
7. Purification of organic acid addition salt as an intermediate is much easier for the purification of the milnacipran base.
8. Since acid addition salts are solids and therefore are easy and safe to handle.
9. Avoiding chromatographic separations.
10. The process is low cost, easy to operate, suitable for industrial scale production.

Those skilled in the art to which the present invention pertains may make modifications resulting in other embodiments employing principles of the present invention without departing from its spirit or characteristics, particularly upon considering the foregoing teachings. Accordingly, the described embodiments are to be considered in all respects only as illustrative, and not restrictive, and the scope of the present invention is, therefore, indicated by the appended claims rather than by the foregoing description or drawings. Consequently, while the present invention has been described with reference to particular embodiments, modifications of structure, sequence, materials and the like apparent to those skilled in the art still fall within the scope of the invention as claimed by the applicant.

What is claimed is:

1. A process for preparation of pharmaceutically acceptable salts of milnacipran comprising the steps of:
a) contacting crude milnacipran with an organic acid in a solvent to obtain organic acid addition salt of said milnacipran of the formula: 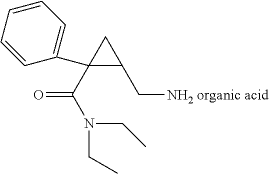
b) contacting said organic acid addition salt with a mineral acid to prepare a mineral acid addition salt without isolating free base of milnacipran of the formula: 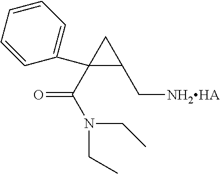
wherein A is anionic part of a mineral acid.

2. The process of claim 1, wherein ‘A’ is selected from the group consisting of Cl, Br, I, HSO₄, H₂PO₄, and NO₃.

3. The process of claim 1, wherein the organic milnacipran acid addition salt and the mineral acid addition salt are racemic.

4. The process of claim 1, wherein milnacipran acid addition salt and the mineral addition salt are (1S,2R)-cis-milnacipran of the formulas:

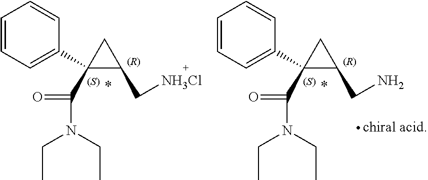

5. The process of claim 1, wherein milnacipran acid addition salt and the mineral addition salt are (1R,2S)-cis-milnacipran of the formulas:

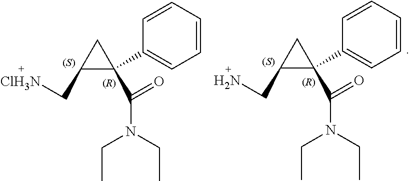

6. The process of claim 1, wherein the organic acid is selected from the group consisting of citric acid, succinic acid, maleic acid, mandelic acid, oxalic acid, tartaric acid, and derivatives thereof.

7. The process of claim 1, wherein the solvent is selected from the group consisting of C1-C4 esters, ketones, aromatic hydrocarbons, ethers and mixture thereof.

8. The process of claim 1, wherein the solvent is selected from the group consisting of methyl acetate, ethyl acetate, propyl acetate, and mixtures thereof.

9. The process of claim 1, wherein the mineral acid is selected from the group consisting of HCl, HBr, HI, sulphuric acid, phosphoric acid and nitric acid.

10. The process of claim 1, wherein the mineral acid is HCl.

11. The process of claim 1, wherein the organic acid addition salt is purified before contacting with the mineral acid to obtain the mineral acid addition salt of said milnacipran.

12. A process for preparation of a substantially pure milnacipran mineral acid addition salt in a single step comprising:
contacting -milnacipran acid addition salt in an organic solvent with a mineral acid directly to obtain milnacipran mineral acid addition salt without isolating free base of the milnacipran of the formula: 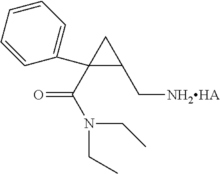
wherein A is anionic part of a mineral acid.

13. The process of claim 12, wherein the organic acid addition salt and the mineral acid addition salt of milnacipran are racemic.

14. The process of claim 12, wherein milnacipran acid addition salt and the mineral acid addition salt are (1S,2R)-cis-milnacipran of the formulas:

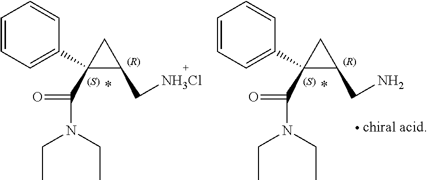

15. The process of claim 12, wherein milnacipran acid addition salt and the mineral acid addition salt are (1R,2S)-cis-milnacipran of the formulas:

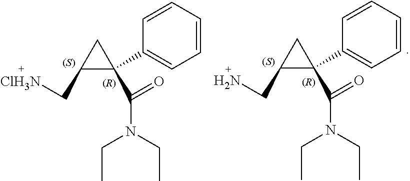

16. The process of claim 12, wherein the organic acid is selected from the group consisting of citric acid, succinic acid, malic acid, mandelic acid, oxalic acid, tartaric acid, and derivatives thereof.

17. The process of claim 12, wherein the solvent is selected from the group consisting of C1-C4 esters, ketones, aromatic hydrocarbons, ethers and mixture thereof.

18. The process of claim 12, wherein the solvent is selected from the group consisting of methyl acetate, ethyl acetate, propyl acetate, and mixtures thereof.

19. The process of claim 12, wherein the mineral acid is selected from the group consisting of HCl, HBr, HI, sulphuric acid, phosphoric acid and nitric acid.

20. A process for preparation of a substantially pure (1S,2R)-cis-milnacipran mineral acid addition salt and/or (1R,2S)-cis-milnacipran mineral acid addition salt in a single step comprising:
contacting (1S,2R)-cis-milnacipran chiral organic acid addition salt or pure (1S,2R)-cis-milnacipran addition salt with a mineral acid to directly obtain (1S,2R)-cis-milnacipran mineral acid addition salt or (1R,2S)-cis-milnacipran without isolating free base of the optically active (1S,2R)-cis-milnacipran or (1R,2S)-cis-milnacipran.
